(19) United States
(12) Patent Application Publication
(10) Pub. No.: US 2008/0219936 A1
(43) Pub. Date: Sep. 11, 2008

(54) Skin depigmenting compositions comprising adapalene, at least one depigmenting agent and at least one anti-inflammatory agent

(75) Inventors: Isabelle Pelisson, Vallauris (FR); Andre Jomard, Saint Vallier De Thiey (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH AND DEVELOPMENT, Biot (FR)

(21) Appl. No.: 12/041,367

(22) Filed: Mar. 3, 2008

Related U.S. Application Data
(63) Continuation of application No. PCT/IB2006/003725, filed on Aug. 13, 2006.

(30) Foreign Application Priority Data
Sep. 2, 2005 (FR) 0509003

(51) Int. Cl. A61K 8/49; A61K 31/192; A61Q 17/04; A61K 31/58

(52) U.S. Cl. 424/59; 514/569; 514/171

(57) ABSTRACT

Depigmenting compositions for the skin, comprise, formulated into a physiologically acceptable medium, adapalene, at least one depigmenting agent and at least one anti-inflammatory agent, and to the pharmaceutical/cosmetic applications thereof.

### CROSS-REFERENCE TO PRIORITY/PCT/PROVISIONAL APPLICATIONS

This application claims priority under 35 U.S.C. § 119 of FR 0509003, filed Sep. 2, 2005, and of Provisional Application No. 60/717,767, filed Sep. 19, 2005, and is a continuation of PCT/IB 2006/003725, filed Aug. 31, 2006 and designating the United States, published in the English language as WO 2007/052157 A2 on May 10, 2007, each hereby expressly incorporated by reference in its entirety and each assigned to the assignee hereof.

### BACKGROUND OF THE INVENTION

1. Technical Field of the Invention

The present invention relates to depigmenting compositions for the skin, comprising formulated into a physiologically acceptable medium, adapalene (6-[3-(1-adamantyl)-4-methoxyphenyl]-2-naphthanoic acid), at least one depigmenting agent and at least one anti-inflammatory bioactive agent, and to various pharmaceutical/cosmetic applications thereof.

2. Description of Background and/or Related and/or Prior Art

A. M. Kligman describes, in U.S. Pat. No. 3,856,934, a depigmenting composition comprising hydroquinone, retinoic acid and a corticosteroid. This type of combination promotes good depigmentation of the skin but elicits substantial adverse effects such as irritation and itching.

Retinoic acid (tretinoin) is known to itself have a skin depigmenting activity (Guevara I. A. and Pandya A. G., Int. J. Dermatol., 40, 210-215 (2001)). In addition, tests carried out on the tail of a mouse not irradiated with UV-B radiation for six weeks show that tretinoin, at concentrations of 0.01% or 0.03%, induces depigmentation of the tail which begins from the first week of treatment and progresses to reach a clear depigmentation at 6 weeks. In mice which are irradiated with UV-B radiation and treated, the dose of 0.03% is capable of inhibiting the induction of pigmentation by UV-B radiation, whereas the lower dose has no effect. Tretinoin is therefore a depigmenting agent in and of itself. On the other hand, under the same experimental conditions, adapalene has no depigmenting activity, as is shown in Example 5 hereinafter. Due to its lack of depigmenting activity in particular, nothing would suggest to one skilled in the art to combine same with a depigmenting agent in a depigmenting composition optionally containing an anti-inflammatory agent.

### SUMMARY OF THE INVENTION

It has now surprisingly been determined that the combination of adapalene, a depigmenting agent and an anti-inflammatory agent elicits a much more rapid depigmenting response than that obtained with the depigmenting agent alone.

It has also now surprisingly been determined that good tolerance of the skin is obtained with the combination of adapalene, a depigmenting agent and an anti-inflammatory agent, while at the same time maintaining a considerable depigmenting activity.

The present invention therefore features depigmenting compositions for the skin, comprising, formulated into a physiologically acceptable medium, adapalene, at least one depigmenting agent other than adapalene, and at least one anti-inflammatory agent also distinct from adapalene. The compositions according to the invention therefore comprise at least three different active ingredients.

The term “physiologically acceptable medium” means a medium which is compatible with the skin, the mucous membranes and/or the integuments thereof.

The term “depigmenting agent” means any active agent having skin-depigmenting activity. This activity makes it possible to decrease the pigmentation of the skin that already exists and also to prevent any additional pigmentation above the natural pigmentation.

### BRIEF DESCRIPTION OF THE DRAWINGS

FIG. 1 is a graph showing the kinetics of certain mouse tail skin pigmentation scores as a function of treatment time with or without UV-B irradiation;

FIG. 2 is a graph showing the scores for pigmentation on mouse tail at the end of a study with certain active agents and with or without UV-B irradiation; and

FIG. 3 is a graph showing the overall clinical score for inflammation on the mouse tail at the end of a study after application of certain species or of a control.

### DETAILED DESCRIPTION OF BEST MODE AND SPECIFIC/PREFERRED EMBODIMENTS OF THE INVENTION

Examples of depigmenting agents, according to the invention include the phenolic derivatives, such as hydroquinone, hydroquinone monoethyl ether, hydroquinone monobenzyl ether or 4-hydroxyanisole, kojic acid and derivatives thereof, azelaic acid and derivatives thereof, linoleic acid, resorcinol and derivatives thereof, ellagic acid, hydroxy acids such as glycolic acid, ascorbic acid, zinc peroxide, and mercury chloride.

In particular, the depigmenting compositions for the skin comprise, as depigmenting agent, a phenolic derivative, in particular hydroquinone.

The present invention thus features depigmenting compositions for the skin, comprising, formulated into a physiologically acceptable medium, adapalene, at least one depigmenting agent distinct from adapalene and at least one anti-inflammatory agent distinct from adapalene.

To provide an order of magnitude, the compositions according to the invention advantageously comprise from 0.0001% to 20% by weight of adapalene relative to the total weight of the composition, and from 0.0001% to 20% by weight of depigmenting agent relative to the total weight of the composition, and preferably, respectively, from 0.001% to 10% by weight of adapalene relative to the total weight of the composition, and from 0.025% to 5% by weight of depigmenting agent relative to the total weight of the composition.

The subject compositions comprise at least one steroidal or non-steroidal anti-inflammatory agent in preferential concentrations ranging from 0.001% to 20.00% by weight relative to the total weight of the composition.

Among the steroidal anti-inflammatory agents, exemplary are clobetasone butyrate, clobetasol propionate, clobetasol dipropionate, hydrocortisone, cortisone, prednisolone, miconazole, prednisone, triamcinolone acetonide, methylprednisolone, fluometholone, fluocinolone acetonide, desonide, betamethasone, dexamethasone, and mixtures thereof.

Among the non-steroidal anti-inflammatory agents, exemplary are indole derivatives, arylcarboxylic derivatives such as ibuprofen, oxicams, pyrazole compounds, salicylic acid and selective COX-2 inhibitors.

In particular, the depigmenting compositions for the skin advantageously comprise fluocinolone acetonide as anti-inflammatory agent.

The subject compositions may also comprise any additive normally employed in the cosmetics or pharmaceutical field, such as sequestering agents, antioxidants, sunscreens, preservatives, fillers, electrolytes, humectants, dyes, common inorganic or organic bases or acids, fragrances, essential oils, cosmetic active agents, moisturizers, vitamins, essential fatty acids, sphingolipids, self-tanning compounds, and skin calmative and protective agents such as allantoin. Of course, one skilled in the art will take care to select this or these optional additional compound(s), and/or the amount thereof, such that the advantageous properties of the compositions according to the invention are not, or are not substantially, adversely affected.

These additives may be present in the composition in a proportion of from 0.001% to 20% by weight relative to the total weight of the composition.

Exemplary sequestering agents include ethylenediaminetetraacetic acid (EDTA), and also derivatives or salts thereof, dihydroxyethylglycine, citric acid and tartaric acid.

Exemplary preservatives include benzalkonium chloride, phenoxyethanol, benzyl alcohol, diazolidinylurea and parabens.

Exemplary humectants include glycerol and sorbitol.

The present invention also features administration of the subject compositions as medicaments.

This invention also features application of the subject compositions in the pharmaceutical and cosmetics fields.

The compositions of the invention are particularly useful, in a regime or regimen, for the treatment and prevention of hyperpigmentary disorders such as melasma, chloasma, lentigines, senile lentigo, freckles, post-inflammatory hyperpigmentations due to an abrasion, a burn, a scar, a dermatosis, a contact allergy; naevi, hyperpigmentations with genetic determinism, hyperpigmentations of metabolic or drug-related origin, melanomas or any other hyperpigmentary lesions.

The compositions according to the invention also find application in the cosmetics field, in particular in protection against the harmful effects of the sun, for preventing and/or for combating photo-induced or chronological aging of the skin and the integuments thereof.

In order to further illustrate the present invention and the advantages thereof, the following specific examples are given, it being understood that same are intended only as illustrative and in nowise limitative. In said examples to follow, all parts and percentages are given as a percentage by weight, unless otherwise indicated.

### Example 1

| | |
|---|---|
| Adapalene | 0.10 |
| Hydroquinone | 4.00 |
| Fluocinolone acetonide | 0.01 |
| Magnesium aluminum silicate | 3.00 |
| Butyl hydroxytoluene | 0.04 |
| Methyl paraben | 0.18 |
| Propyl paraben | 0.02 |
| Cetyl alcohol | 4.00 |
| Stearic acid | 3.00 |
| Stearyl alcohol | 4.00 |
| Glyceryl stearate/PEG-100 stearate | 3.50 |
| Methyl gluceth-10 | 5.00 |
| Glycerol | 4.00 |
| Citric acid | 0.05 |
| Sodium metabisulfite | 0.20 |
| Purified water | qs 100 |

This composition should be applied in a regime or once a day until complete depigmentation is obtained, for the treatment of chloasma.

### Example 2

| | |
|---|---|
| Adapalene | 0.10 |
| 4-Hydroxyanisole | 3.00 |
| Fluocinolone acetonide | 0.01 |
| Magnesium aluminum silicate | 3.00 |
| Butyl hydroxytoluene | 0.04 |
| Methyl paraben | 0.18 |
| Propyl paraben | 0.02 |
| Cetyl alcohol | 4.00 |
| Stearic acid | 3.00 |
| Stearyl alcohol | 4.00 |
| Glyceryl stearate/PEG-100 stearate | 3.50 |
| Methyl gluceth-10 | 5.00 |
| Glycerol | 4.00 |
| Citric acid | 0.05 |
| Sodium metabisulfite | 0.20 |
| Purified water | qs 100 |

This composition should be applied twice a day until complete depigmentation is obtained, for the treatment of lentigines.

### Example 3

| | |
|---|---|
| Adapalene | 0.1 |
| Hydroquinone | 2 |
| Desonide | 0.05 |
| Carbomer | 0.1 |
| Methyl paraben | 0.18 |
| Propyl paraben | 0.02 |
| Cetyl alcohol | 1 |
| Stearic acid | 0.8 |
| Caprylic-capric triglyceride | 1.5 |
| Cyclomethicone | 1 |
| Mineral oil | 2 |
| Propylene glycol | 5 |
| Glycerol | 2 |
| Triethanolamine | 5 |
| Citric acid (qs pH 5.5-6.0) | / |
| Purified water | qs 100 |

This composition should be applied once a day until complete depigmentation is obtained, for the treatment of melasma.

### Example 4

| | |
|---|---|
| Adapalene | 0.1 |
| 4-Hydroxyanisole | 2 |
| Salicylic acid | 1 |
| Steareth-2 | 3 |
| Steareth-21 | 2 |
| Caprylic-capric triglyceride | 4 |
| Isohexadecane | 5 |
| Cetearyl alcohol | 1 |
| Stearic acid | 1.5 |
| Sodium sulfite | 0.2 |
| Propylene glycol | 8 |
| Glycerol | 2 |
| Phenoxyethanol | 1 |
| Citric acid (qs pH 5.5-6.0) | / |
| Purified water | qs 100 |

This composition should be applied twice a day until complete depigmentation is obtained, for the treatment of lentigines.

### Example 5

### Measurement of the Depigmenting Activity of the Combination of Adapalene and a Depigmenting Agent

The evaluation of the depigmenting and/or anti-pigmenting activity of 3% hydroquinone and of 0.1% adapalene, alone or in combination for 8 weeks, is carried out on the tail of an SKH:HR2 mouse irradiated with ultraviolet B radiation, or not irradiated. The tail of the SKH:HR2 mouse is naturally pigmented and this pigmentation increases under the effect of repeated UV-B irradiation. The depigmenting activity is measured on the natural pigmentation after application of the test product to the tail of non-irradiated animals. The anti-pigmenting activity is measured by the inhibition of the induction of the UV-induced pigmentation: the test product is applied to the tail of UV-B-irradiated animals.

The treatment is carried out 5 days a week for 8 weeks. 20 μl of test product diluted in acetone are applied to the tail with a gap from applications: the hydroquinone is applied in the morning and the adapalene 4 h later. On the days of irradiation, the treatment is applied after irradiation.

The animals are irradiated 3 times a week for 8 weeks (Monday, Wednesday, Friday) at the dose of 90 mJ/cm² of UV-B.

The pigmentation is evaluated once a week before irradiation, by means of a score on a scale of 0 to 4. The scores are divided as follows: Depigmentation scale: scores −1 to −4.

0: natural pigmentation

−1: slight depigmentation

−2: moderate depigmentation

−3: marked depigmentation

−4: complete depigmentation

Pigmentation scale: scores 1 to 4

1: slight pigmentation

2: moderate pigmentation

3: marked pigmentation

4: strong pigmentation

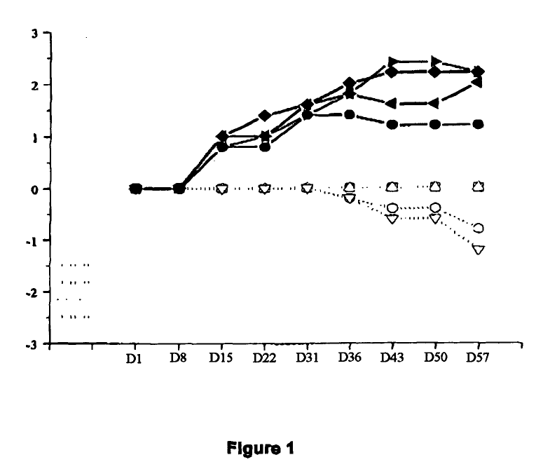

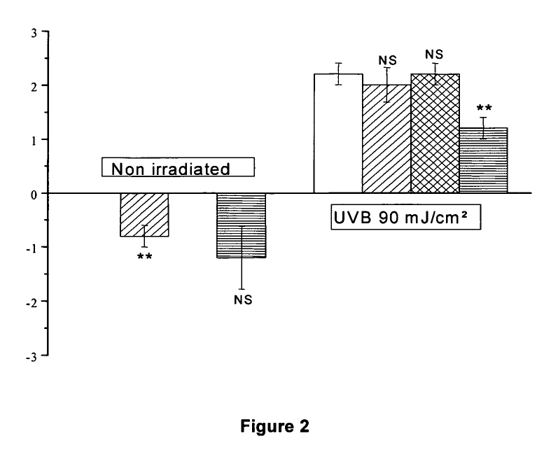

The results are shown in FIGS. 1 and 2.

FIG. 1 represents the kinetics of the mouse tail skin pigmentation scores as a function of treatment time with or without ultraviolet B irradiation (up to 8 weeks of treatment) with (♦) UV-B+acetone, [symbol] UV-B+adapalene, [symbol] UV-B+hydroquinone, () UV-B+hydroquinone+adapalene, (□) skin not irradiated+acetone, (Δ) skin not irradiated+adapalene, (∘) skin not irradiated+hydroquinone, (∇) skin not irradiated+hydroquinone+adapalene.

FIG. 2 represents the scores for pigmentation on the tail at the end of the study (D57) with (□) acetone, [symbol] hydroquinone, [symbol] adapalene, [symbol] adapalene+hydroquinone with or without ultraviolet B irradiation.

Depigmenting Activity:

Hydroquinone alone at 3% induces a depigmentation which is clinically visible from the 6th week of treatment (D43) and statistically significant at the end of the study. Adapalene alone does not modify the natural pigmentation. When adapalene is combined with hydroquinone, it potentiates its depigmenting activity.

Anti-Pigmenting Activity:

In the animals irradiated and treated concomitantly, hydroquinone shows an anti-pigmenting effect at the 6th and at the 7th week (D50), which becomes less marked at the end of the study. On the other hand, the adapalene+hydroquinone combination inhibits the pigmentation induced by UV-B irradiation as soon as it appears and until the end of the study, where the difference is statistically significant (**, p<0.01).

Conclusion:

After 8 weeks of topical treatment on the tail of an SKH:HR2 mouse, it is noted that the hydroquinone+adapalene combination exhibits a strong anti-pigmenting activity and significantly inhibits the pigmentation induced by UV-B irradiation as soon as it appears and until the end of the study. The hydroquinone+adapalene combination exhibits a significant benefit as a depigmenting agent, with respect to hydroquinone alone.

### Example 6

### Measurement of the Tolerance with Respect to the Combination of Adapalene, an Anti-Pigmenting Agent and an Anti-Inflammatory Agent

The evaluation of the tolerance with respect to the adapalene+hydroquinone+fluocinolone acetonide combination (“adapalene trio”) formulated in a cream, in comparison with that obtained with the combination retinoic acid (tretinoin)+hydroquinone+fluocinolone acetonide (“control trio”) in the same vehicle for 4 weeks, is carried out on the tail of an SKH:HR2 mouse which is UV-B-irradiated or not irradiated. The same measurements are also conducted on the vehicle alone and without active agents. The tolerance after application of the test product is measured with ultraviolet B (UBV)-irradiation and without ultraviolet B-irradiation.

The treatment is carried out 5 days a week for 4 weeks. 20 μl of formulated test product are applied to the tail. On the days of irradiation, the treatment is applied after irradiation.

The animals are irradiated 3 times a week for 4 weeks (Monday, Wednesday, Friday) at the dose of 90 mJ/cm² of UV-B.

The inflammation (erythema and squamae) is evaluated once a week before irradiation by means of a score on a scale of 0 to 4. The scores are divided as follows:

0: normal skin without inflammation

1: slight inflammation

2: moderate inflammation

3: marked inflammation

4: severe inflammation.

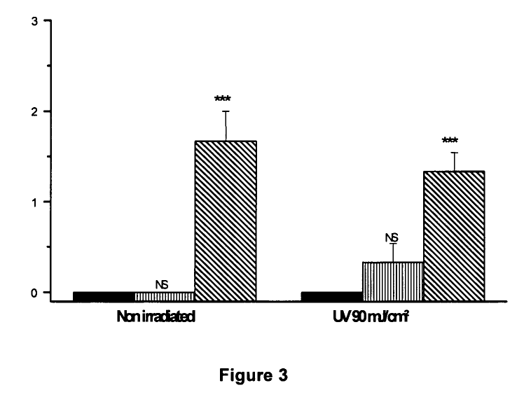

The results are shown in FIG. 3.

FIG. 3 presents the overall clinical score for inflammation on the tail at the end of the 4 weeks of study after application either of the vehicle [symbol] or of the adapalene trio [symbol] or of the control trio [symbol] .

Conclusion:

The signs of inflammation are clearly less marked with the adapalene trio than with the control trio.

The comparative study of adapalene trio versus control trio for 4 weeks on the tail in mice shows that the adapalene trio exhibits much better tolerance than the control trio.

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A topically applicable skin depigmenting composition, comprising adapalene, at least one depigmenting agent other than adapalene and at least one anti-inflammatory agent other than adapalene, formulated into a topically applicable physiologically acceptable vehicle therefor.

2. The depigmenting composition as defined by claim 1, said at least one depigmenting agent comprising a phenolic derivative.

3. The depigmenting composition as defined by claim 1, said at least one depigmenting agent comprising hydroquinone.

4. The depigmenting composition as defined by claim 1, said at least one anti-inflammatory agent comprising fluocinolone acetonide.

5. The depigmenting composition as defined by claim 1, comprising a medicament.

6. A regime or regimen for the prevention or treatment of a hyperpigmentary disorder, melasma, chloasma, lentigines, freckles, a post-inflammatory hyperpigmentation due to an abrasion, a burn, a scar, a dermatosis, a contact allergy, naevi, a hyperpigmentation with a genetic determinism, a hyperpigmentation of metabolic or drug-related origin, a melanoma or other hyperpigmentary lesion, comprising topically administering to a subject in need of such treatment, a thus effective amount of the depigmenting composition as defined by claim 1.

7. A regime or regimen for the prevention or treatment of melasma, comprising topically administering to a subject in need of such treatment, a thus effective amount of the depigmenting composition as defined by claim 1.

8. A regime or regimen for protecting the skin and/or integuments against the harmful effects of the sun, for preventing and/or for combating photo-induced or chronological aging thereof, comprising topically administering to a subject in need of such treatment, a thus effective amount of the depigmenting composition as defined by claim 1.

9. The depigmenting composition as defined by claim 1, said at least one anti-inflammatory agent comprising a steroidal anti-inflammatory agent.

10. The depigmenting composition as defined by claim 1, said at least one anti-inflammatory agent comprising a non-steroidal anti-inflammatory agent.

11. The depigmenting composition as defined by claim 1, comprising from 0.0001% to 20% by weight of adapalene.

12. The depigmenting composition as defined by claim 11, comprising from 0.0001% to 20% by weight of said at least one depigmenting agent.

13. The depigmenting composition as defined by claim 12, comprising from 0.0001% to 20% by weight of said at least one anti-inflammatory agent.
